(19) United States
(12) Patent Application Publication
(10) Pub. No.: US 2009/0060855 A1
(43) Pub. Date: Mar. 5, 2009

(54) Bleaching composition comprising a non-volatile liquid branched ester of carboxylic acid with solidification point below 4&deg; C

(76) Inventors: Benoit Boche, La Garenne Colombes (FR); Damarys Braida-Valerio, Paris (FR); Luc Nicolas-Morgantini, Rully (FR); Sylvain Kravtchenko, Shanghai (CN)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/007,722

(22) Filed: Jan. 15, 2008

Related U.S. Application Data
(60) Provisional application No. 60/897,501, filed on Jan. 26, 2007.

(30) Foreign Application Priority Data
Jan. 15, 2007 (FR) 0752677

(51) Int. Cl. A61K 8/22

(52) U.S. Cl. 424/62

(57) ABSTRACT

The present disclosure relates to compositions for the bleaching of keratinous fibers comprising at least one peroxygenated salt and at least one non-volatile liquid branched ester of a carboxylic acid, the solidification point of which is below 4° C., chosen from the compounds with the following structure: R₁—CO—O—R₂, wherein R₁ and R₂ are each independently chosen from a C₁-C₃₀ hydrocarbon chain optionally interrupted by at least one oxygen atom and/or by at least one carbonyl group and optionally substituted by at least one hydroxyl groups, R₁ being branched. The composition disclosed herein for the bleaching of keratinous fibers exhibits an improved resistance to low temperatures and makes it possible to avoid the problem of syneresis during storage at low temperatures and during transportation in which temperature cycles occur.

This application claims benefit of U.S. Provisional Application No. 60/897,501, filed Jan. 26, 2007, the contents of which are incorporated herein by reference. This application also claims benefit of priority under 35 U.S.C. § 119 to French Patent Application No. FR 0752677, filed Jan. 15, 2007, the contents of which are also incorporated herein by reference.

Disclosed herein, in at least one aspect, is a composition for bleaching keratinous fibers, such as human keratinous fibers, for example the hair, comprising at least one peroxygenated salt and at least one non-volatile liquid branched ester of a carboxylic acid with a solidification point below 4° C.

The bleaching of human keratinous fibers, for example the hair, takes place by oxidation of the pigment “melanin,” resulting in the dissolution and removal, partial or complete, of this pigment.

To bleach hair, bleaching powders comprising a peroxygenated reactant, such as ammonium or alkali metal persulphates, perborates and percarbonates, which are combined at the time of use with an aqueous hydrogen peroxide composition, are most often used.

Because peroxygenated salts and hydrogen peroxide are relatively stable in an acidic medium, it is often necessary to activate them at a basic pH in order to obtain appropriate formation of oxygen. Thus, it is normal to add alkaline compounds, such as urea, alkali metal or alkaline earth metal silicates and phosphates, for example alkali metal metasilicates, or agents which are precursors of ammonia, such as ammonium salts, to the bleaching powders.

However, bleaching powders may have a tendency to form dust during their handling, transportation and storage thereof.

In addition, the ingredients forming these bleaching powders (persulphates, alkaline silicates) may be corrosive and irritating to the eyes, respiratory tract and mucous membranes.

To address these issues, pastes have been developed which comprise the pulverulent agents (e.g., peroxygenated salts, alkaline agents, thickeners) in an inert liquid organic vehicle. Such compositions are described, for example in German Patent Application Nos. DE 38 14 356 and DE 197 23 538.

However, current bleaching pastes based on this technology may have unsatisfactory physicochemical stability, and do not always make it possible to obtain sufficiently homogeneous and powerful bleaching. In addition, they do not always have an attractive appearance.

To address the known stability problems of bleaching pastes, combinations of certain thickening agents have been explored. Such combinations are described, for example in European Patent Application Nos. EP 0 778 020 and EP 1 034 777.

To further enhance the stability of bleaching paste compositions, use has also been made of waxes that thicken the inert organic liquid. As used herein, the term, “waxes,” means products having a melting point greater than 40° C., esters of fatty acids which are hydrophobic and which comprise a long chain, and substitution products of beeswax.

However, in order to be dispersed and/or dissolved in the organic liquid, these waxes must be molten, which implies that they are heated during the manufacturing process.

In addition, such compositions based on waxes may be sensitive to temperature and to thermal impacts, both during their manufacture and their storage. In such instances, the pastes may lose their usual qualities.

In order to overcome these disadvantages, the proposal has been made, in French Patent Application Nos. FR 2 842 099 and FR 2 842 100, to use a combination of an inert organic liquid and a pyrogenic silica having a hydrophilic or hydrophobic nature, or a combination of a polydecene and a gelling agent chosen from pyrogenic silicas having a hydrophilic or hydrophobic nature and diblock, triblock, multiblock or radical block copolymers composed of segments of styrene monomer type and of segments of thermoplastic monomer or comonomer type.

However, the bleaching pastes currently available commercially may still exhibit the disadvantage of not being very resistant to cold temperatures. In particular, such pastes may exhibit syneresis problems, i.e., exudation of the oily phase, when stored at low temperatures, as well as during their transportation in which cyclic temperature changes can occur.

Thus, it would be desirable to provide a composition for the bleaching of keratinous fibers which addresses the problem of volatility of the powders, while avoiding the disadvantages of the compositions of the prior art. In particular it would be desirable to provide a composition for the bleaching of keratinous fibers that exhibits good resistance to low temperatures, while making it possible to obtain powerful and homogeneous bleaching without leaving the hair greasy or rough.

The present inventors have found that the compositions of the present disclosure solve at least one of the above-discussed disadvantages. Disclosed herein, therefore, are compositions for the bleaching of keratinous fibers comprising at least one peroxygenated salt and at least one non-volatile liquid branched ester of a carboxylic acid, the solidification point of which is below 4° C., and which is chosen from compounds with the following structure (I):

R₁—CO—O—R₂ (I)

wherein R₁ and R₂, are each independently chosen from a C₁-C₃₀, for example, a C₂-C₂₀ hydrocarbon chain optionally interrupted by at least one oxygen atom and/or by at least one carbonyl group, and optionally substituted by at least one hydroxyl group, and wherein R₁ is branched.

Other aspects of the present disclosure include a method for bleaching keratinous fibers, comprising applying a composition in accordance with the present disclosure to keratinous fibers, as well as a multicompartment device for the application of such compositions.

Another aspect of the present disclosure relates to the use of a non-volatile liquid branched ester of a carboxylic acid, the solidification point of which is below 4° C., as described above, in a composition for the bleaching of keratinous fibers comprising a peroxygenated salt.

The present disclosure makes it possible to obtain a composition for the bleaching of keratinous fibers which exhibits an improved resistance to low temperatures, and which, in at least one embodiment, makes it possible to avoid the problem of syneresis during storage at low temperatures and during transportation in which temperature cycles occur.

Unless otherwise indicated, the limits of the ranges of values which are given in the context of the present disclosure are included within these ranges.

The at least one peroxygenated salt present in the composition in accordance with the disclosure may, for example, be chosen from persulphates, perborates, percarbonates or peroxides of alkali metals or alkaline earth metals, and mixtures thereof. For example, the at least one peroxygenated salt may be chosen from persulphates and mixtures thereof, such as sodium persulphate, potassium persulphate, and ammonium persulphate, and mixtures thereof.

The at least one peroxygenated salt may be present in the compositions according to the present disclosure in an amount ranging from 10% to 70% by weight, for example, from 20% to 60% by weight, relative to the total weight of the composition.

As used herein, the term “low temperature,” means a temperature below 10° C., for example, below 5° C.

As used herein, the term “liquid,” means any phase capable of flowing under the action of its own weight at ambient temperature, e.g., from 15° C. to 40° C., and at atmospheric pressure.

As used herein, the term “non-volatile,” means a compound exhibiting a vapor pressure less than or equal to 5 mmHg at a temperature of 20° C. In at least one embodiment of the present disclosure, the vapor pressure is less than 1 mmHg.

As used herein, the term “branched ester,” means an ester comprising, in the part resulting from an acid and/or in the part resulting from an alcohol, at least one branched hydrocarbon chain comprising at least three carbon atoms.

The non-volatile liquid branched ester or esters of carboxylic acids of use in the context of the present disclosure have a solidification temperature below 4° C. This solidification temperature may be determined, for example, by DSC (differential scanning calorimetry). Mention may be made, as a DSC device which can be used, of the Pyris 1 device from Perkin-Elmer.

In at least one non-limiting embodiment, the non-volatile liquid branched ester or esters of carboxylic acids having a solidification point below 4° C. result from a branched acid.

In at least one non-limiting embodiment of the present disclosure, the non-volatile liquid branched ester or esters of carboxylic acids having a solidification point below 4° C. comprise at least 8 carbon atoms.

As examples of esters that can be used in the present disclosure, non-limiting mention is made of octyl isononanoate, isononyl isononanoate, isobutyl isobutyrate or 2,2,4-trimethyl-1,3-pentanediol butyrate.

In at least one non-limiting embodiment, R₁ and R₂ in the compounds of formula I are both branched. In such case, the non-volatile liquid branched ester or esters of carboxylic acids having a solidification point below 4° C. result from a branched acid and a branched alcohol.

In at least one non-limiting embodiment, isononyl isononanoate is used as the at least one non-volatile liquid branched ester.

The non-volatile liquid branched esters of carboxylic acids having a solidification point below 4° C. may be present in the composition according to the disclosure in an amount ranging from 1 to 70% by weight, for example, from 5 to 60% by weight, such as from 10% to 50% by weight, relative to the total weight of the composition.

In some embodiments, the compositions in accordance with the present disclosure further comprise at least one alkaline agent.

The at least one alkaline agent or agents may, for example, be chosen from urea, ammonium salts, such as ammonium chloride, ammonium sulphate, ammonium phosphate or ammonium nitrate, silicates, phosphates or carbonates of alkali metals or alkaline earth metals, such as lithium, sodium, potassium, magnesium, calcium or barium, and mixtures thereof. In some embodiments, the at least one alkaline agent is chosen from silicates, carbonates, ammonium chloride and mixtures thereof.

When present in the composition in accordance with the present disclosure, the alkaline agent(s) may be present in an amount ranging from 0.01 to 40% by weight, for example, from 0.1 to 30% by weight, relative to the total weight of the composition.

In at least one non-limiting embodiment, the composition of the present disclosure further comprises at least one additional inert organic liquid other than the non-volatile liquid branched esters of carboxylic acids having a solidification temperature below 4° C.

As used herein, the term “inert organic liquid,” means, an organic liquid which is chemically inert with regard to hydrogen peroxide. In the context of the present disclosure, a liquid is inert if the decomposition of hydrogen peroxide in the presence of the liquid is less than 25% after 15 hours at 100° C.

As examples of inert organic liquids that may be used, non-limiting mention may be made of polydecenes of the formula C₁₀ₙH_{[(20n)+2]}, wherein n ranges from 3 to 9, for example from 3 to 7, esters of fatty alcohols or of fatty acids other than those described above, esters or diesters of sugars and of C₁₂-C₂₄ fatty acids, cyclic ethers or cyclic esters, silicone oils, mineral oils, vegetable oils, and mixtures thereof.

The compounds of formula C₁₀ₙH_{[(20n)+2]} with n varying from 3 to 9 correspond to the term “polydecene,” in the CTFA Dictionary, 7th edition, 1997, of the Cosmetic, Toiletry and Fragrance Association, USA, and to the same INCI term in the USA and Europe. These products may, for example, be produced by the hydrogenation of poly-1-decenes.

In some embodiments, polydecenes of the formula C₁₀ₙH_{[(20n)+2]}, where n varies from 3 to 7, are used.

As examples of such polydecenes, non-limiting mention may be made of the products sold under the name Silkflo® 366 NF Polydecene by Amoco Chemical, or those sold under the name Nexbase® 2002 FG, 2004 FG, 2006 FG and 2008 FG, by Fortum.

As non-limiting examples of the esters of fatty alcohols or of fatty acids other than the esters disclosed herein, mention may be made of:

esters of saturated, linear or branched, lower C₃-C₆ monoalcohols with monofunctional C₁₂-C₂₄ fatty acids, wherein said fatty acids may be saturated or unsaturated, linear or branched, and chosen, in at least one embodiment, from oleates, laurates, palmitates, myristates, behenates, cocoates, stearates, linoleates, linolenates, caprates, arachidonates, and mixtures thereof, for example, oleopalmitates, oleostearates, palmitostearates, and the like. Further non-limiting mention may be made of isopropyl palmitate and isopropyl myristate.

esters of linear or branched, C₃-C₈ monoalcohols with bifunctional C₈-C₂₄ fatty acids, wherein said bifunctional C₈-C₂₄ fatty acids may be saturated or unsaturated and linear or branched,

esters of a trifunctional acid.

As used herein with respect to the esters and diesters of sugars and of C₁₂-C₂₄ fatty acids, the term “sugar” means compounds having several alcohol functional groups, with or without an aldehyde or ketone functional group, and which comprise at least 4 carbon atoms. These sugars may be monosaccharides, oligosaccharides or polysaccharides.

As examples of such sugars, non-limiting mention is made of sucrose, glucose, galactose, ribose, fuctose, maltose, fructose, mannose, arabinose, xylose, lactose, and derivatives thereof. In some embodiments, alkylated derivatives, such as methylated derivatives, for example methylglucose, are used.

The esters of sugars and of fatty acids which can be used according to the present disclosure may be chosen, for example, from esters or mixtures of esters of sugars described above and of C₁₂-C₂₄ fatty acids which are saturated or unsaturated and linear or branched.

The esters may be chosen, for example from mono-, di-, tri- and tetraesters, polyesters and mixtures thereof.

These esters may, for example, be chosen from oleates, laurates, palmitates, myristates, behenates, cocoates, stearates, linoleates, linolenates, caprates, arachidonates and mixtures thereof, such as, oleopalmitate, oleostearate or palmitostearate mixed esters.

Further non-limiting mention is made of the use of mono- and diesters, such as mono- or dioleates, -stearates, -behenates, -oleopalmitates, -linoleates, -linolenates or -oleostearates, of sucrose, of glucose or of methylglucose.

Silicone oils may also be employed as an inert organic liquid.

In some embodiments, the silicone oils are chosen from liquid and non-volatile silicone fluids having a viscosity of less than or equal to 10 000 mPa·s at 25° C., the viscosity of the silicones being measured according to Standard ASTM 445 Appendix C.

Silicone oils are described in more detail in the work by Walter Noll, “Chemistry and Technology of Silicones”, 1968, Academic Press.

As examples of silicone oils that may be used, non-limiting mention may be made of the silicone oils sold under the names DC 200 Fluid—5 mPa·s, DC 200 Fluid—20 mPa·s, DC 200 Fluid—350 mPa·s, DC 200 Fluid—1000 mPa·s and DC 200 Fluid—10 000 mPa·s by Dow Corning.

Mineral oils, such as liquid paraffin, may also be used as an inert organic liquid.

Vegetable oils, such as avocado oil, olive oil or jojoba liquid wax, may also be used as an inert organic liquid.

In some embodiments, the at least one additional inert organic liquid is chosen from polydecenes of formula C₁₀ₙH_{[(20n)+2]}, wherein n ranges from 3 to 9, for example 3 to 7, esters of fatty alcohols or of fatty acids other than the esters described above, and mixtures thereof.

The at least one additional inert organic liquid may be present, for example, in an amount ranging from 5% to 60% by weight, such as from 10% to 50% by weight, for example from 15% to 45% by weight, relative to the total weight of the composition.

In some embodiments, the compositions according to the present disclosure are provided in the form of an anhydrous paste.

In the context of the present disclosure, a composition is anhydrous when it has a water content of less than 1% by weight, such as less than 0.5% by weight, relative to the total weight of the composition.

In some embodiments, the composition in accordance with the present disclosure further comprises hydrogen peroxide. In that case, the composition is ready for use and results from the mixing of a composition provided in the form of an anhydrous paste in accordance with the disclosure with an aqueous composition comprising hydrogen peroxide. Its pH may range, for example from 3 to 11, such as from 7 to 11.

The composition in accordance with the present disclosure may also comprise various additives conventionally used in cosmetics.

As non-limiting examples of such additives, mention may be made of: inorganic or organic thickening agents, such as anionic, cationic, nonionic or amphoteric, associative or nonassociative, thickening polymers; fillers, such as clays; binders, such as vinylpyrrolidone; lubricating agents, such as polyol stearates or alkali metal or alkaline earth metal stearates; hydrophilic or hydrophobic silicas; pigments; dyes; mattifying agents; such as titanium oxides; anionic, nonionic, cationic, amphoteric or zwitterionic surface-active agents; antioxidizing agents; penetrating agents; sequestering agents; buffers; dispersing agents; film-forming agents; preservatives; opacifying agents; vitamins, fragrances; anionic; cationic; nonionic; amphoteric or zwitterionic polymers; ceramides; and conditioning agents, such as, for example, volatile or non-volatile and modified or unmodified silicones.

When the composition in accordance with the disclosure comprises hydrogen peroxide, it may also comprise additives and agents for controlling the release of oxygen, such as magnesium carbonate or magnesium oxide.

The additives and agents for controlling the release of oxygen as described above may each be present, for example, in an amount ranging from 0.01 to 40% by weight, such as from 0.1 and 30% by weight, relative to the total weight of the composition.

Of course, a person skilled in the art will take care to choose optional additional compounds so that the advantageous properties intrinsically attached to the composition in accordance with the disclosure are not, or not substantially, detrimentally affected.

The bleaching process in accordance with the present disclosure comprises applying, to the keratinous fibers, in the presence of an aqueous composition comprising hydrogen peroxide, a composition according to the present disclosure, wherein said composition is provided in the form of an anhydrous paste.

The aqueous composition comprising hydrogen peroxide may, for example, be added to the composition provided in the form of an anhydrous paste at the moment of use. It may also be applied simultaneously with or sequentially to the composition provided in the form of an anhydrous paste.

Another aspect of the present disclosure is a multicompartment device comprising at least two compositions packaged separately, the mixing of which results in a composition comprising hydrogen peroxide in accordance with the present disclosure as defined above.

In some embodiments, the multicompartment device in accordance with the present disclosure comprises a first compartment comprising a composition in the form of an anhydrous paste in accordance with the present disclosure as defined above, and a second compartment comprising an aqueous composition comprising hydrogen peroxide.

The cosmetic medium of the aqueous composition comprising hydrogen peroxide may comprise water or a mixture of water and at least one organic solvent, in order to resolve the compounds which would be insufficiently soluble in the water. Mention may be made, as suitable organic solvents, for example, of lower C₁-C₄ alkanols, such as ethanol and isopropanol; glycerol; glycols and glycol ethers, such as 2-butoxyethanol, propylene glycol or propylene glycol monomethyl ether, and aromatic alcohols, such as benzyl alcohol or phenoxyethanol, analogous products and mixtures thereof.

The solvent(s) may be present in an amount ranging from 1 to 40% by weight, for example, from 5 to 30% by weight, relative to the total weight of the dyeing composition.

In some embodiments, the aqueous composition comprising hydrogen peroxide exhibits a pH of less than 7, so as to guarantee the stability of the hydrogen peroxide in this composition.

The aqueous composition comprising hydrogen peroxide may be provided in various forms, such as in the form of a liquid, cream, gel, or in any other form appropriate for carrying out bleaching of keratinous fibers.

The aqueous composition comprising hydrogen peroxide may also include various additives conventionally used in cosmetics, such as those described above.

The aqueous composition comprising hydrogen peroxide can also comprise agents for controlling the release of oxygen as defined above.

The device in accordance with the present disclosure may be equipped with a means for delivering the desired mixture to the hair. Non-limiting examples of such means include the devices described in French Patent No. FR 2 586 913.

With a device according to the present disclosure, it is possible to bleach keratinous fibers with a process in accordance with the disclosure, as described above.

Another aspect of the present disclosure is the use of at least one non-volatile liquid branched ester of a carboxylic acid having a solidification point below 4° C. as defined above in a composition for the bleaching of keratinous fibers comprising at least one peroxygenated salt.

In at least one non-limiting embodiment, the use in accordance with the disclosure makes it possible to improve the resistance to low temperatures of the composition for the bleaching of keratinous fibers, and makes it possible to avoid the problems of syneresis during storage at low temperatures and during transportation in which temperature cycles occur.

The disclosure will be more fully illustrated using the following non-limiting examples.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The amounts of the ingredients are expressed as percentages by weight, relative to the total weight of the composition.

### EXAMPLES

The following anhydrous bleaching pastes were prepared:

| | A | B |
|---|---|---|
| Composition | (Invention) | (Prior art) |
| Sodium persulphate | 5.91 g | 5.91 g |
| Sodium disilicate hydrate | 12.78 g | 12.78 g |
| Potassium persulphate | 36 g | 36 g |
| Ethylenediaminetetraacetic acid | 0.17 g | 0.17 g |
| Pyrogenic silica with a hydrophilic nature | 1.75 g | 1.75 g |
| Titanium oxide | 0.34 g | 0.34 g |
| Isopropyl myristate | 0.64 g | 33.78 g |
| White beeswax | 0.1 g | 0.1 g |
| Isononyl isononanoate | 33.14 g | — |
| N-Oleoyldihydrosphingosine | 0.01 g | 0.01 g |
| Guar gum | 0.85 g | 0.85 g |
| Hydroxyethyl cellulose | 0.64 g | 0.64 g |
| Carboxymethyl potato starch | 2.56 g | 2.56 g |
| Sodium lauryl sulphate | 3.41 g | 3.41 g |
| Magnesium stearate | 1.7 g | 1.7 g |

Each of the bleaching pastes described above was subjected to various tests in order to evaluate its resistance to cold and to transportation.

### Test 1

Each of the bleaching pastes described above was placed in a refrigerator at 4° C. for one week. After returning to ambient temperature, the following points were observed:

composition A did not exhibit any modification;

in the case of composition B, the hardening of the paste and then the formation of pockets of oil were observed.

### Test 2

Each of the bleaching pastes described above was subjected to two temperature cycles from 20° C. to −20° C. (one cycle =6 hours at 20° C., then from 20° C. to −20° C. in 6 hours, then 6 hours at −20° C., then from −20° C. to 20° C. in 6 hours), followed by stirring for 1 hour, which simulated the vibrations undergone by a sample during transportation by lorry over a distance of 1000 km. The following points were observed:

composition A did not exhibit any modification;

in the case of composition B, phase separation of oil at the surface of the paste was observed.

### Test 3

The thermal behavior during cooling from 25° C. to −40° C. at a rate of 5° C./minute was recorded by differential scanning calorimetry for each of the bleaching pastes described above. The following points were observed:

composition A did not show any transition;

composition B exhibited a pronounced crystallization peak beginning at −4° C.

In conclusion, a clear superiority for resistance to cold and to transportation of the anhydrous paste comprising isononyl isononanoate in comparison with the anhydrous paste comprising isopropyl myristate was observed.

What is claimed is:

1. A composition for the bleaching of keratinous fibers, comprising;
at least one peroxygenated salt; and
at least one non-volatile liquid branched ester of a carboxylic acid, the solidification point of which is below 4° C., and which is chosen from compounds of formula (I):
R₁—CO—O—R₂ (I)
wherein R₁ and R₂ are independently chosen from a C₁-C₃₀, hydrocarbon chain optionally interrupted by at least one oxygen atoms and/or by at least one carbonyl group and optionally substituted by at least one hydroxyl group, and wherein R₁ is branched.

2. A composition according to claim 1, wherein said at least one peroxygenated salt is chosen from persulphates, perborates, percarbonates or peroxides of alkali metals or alkaline earth metals, and mixtures thereof.

3. A composition according to claim 2, wherein said at least one peroxygenated salt is chosen from persulphates and mixtures thereof.

4. A composition according to claim 1, wherein said at least one peroxygenated salt is present in said composition in an amount ranging from 10% and 70% by weight, relative to the total weight of the composition.

5. A composition according to claim 1, wherein said at least one non-volatile liquid branched ester comprises at least 8 carbon atoms.

6. A composition according to claim 1, wherein both R₁ and R₂ are branched.

7. A composition according to claim 1, wherein said at least one non-volatile liquid branched ester is isononyl isononanoate.

8. A composition according to claim 1, wherein said at least one non-volatile liquid branched ester is present in an amount ranging from 1% and 70% by weight, relative to the total weight of the composition.

9. A composition according to claim 1, further comprising at least one alkaline agent.

10. A composition according to claim 9, wherein said at least one alkaline agent is chosen from urea, ammonium salts, silicates, phosphates or carbonates of alkali metals or alkaline earth metals, and mixtures thereof.

11. A composition according to claim 10, wherein said at least one alkaline agent is chosen from silicates, carbonates, ammonium chloride and mixtures thereof.

12. A composition according to claim 9, wherein said at least one alkaline agent is present in an amount ranging from 0.01 to 40% by weight, relative to the total weight of the composition.

13. A composition according to claim 1, further comprising at least one additional inert organic liquid other than said non-volatile liquid branched esters of carboxylic acids having a solidification point below 4° C.

14. A composition according to claim 13, wherein said at least one additional inert organic liquid is chosen from polydecenes of formula C₁₀ₙH_{[(20n)+2]}, wherein n ranges from 3 to 9, esters of fatty alcohols or of fatty acids other than non-volatile liquid branched esters of carboxylic acids having a solidification point below 4° C., esters or diesters of sugars and of C₁₂-C₂₄ fatty acids, cyclic ethers or cyclic esters, silicone oils, mineral oils, and vegetable oils, and mixtures thereof.

15. A composition according to claim 13, wherein said at least one additional inert organic liquid is present in an amount ranging from 5% to 60% by weight, relative to the total weight of the composition.

16. A composition according to claim 1, wherein said composition is in the form of an anhydrous paste.

17. A composition according to claim 1, further comprising hydrogen peroxide.

18. A bleaching process, comprising applying to keratinous fibers, in the presence of an aqueous composition comprising hydrogen peroxide, a composition in the form of an anhydrous paste comprising:
at least one peroxygenated salt; and
at least one non-volatile liquid branched ester of a carboxylic acid, the solidification point of which is below 4° C., and which is chosen from compounds of formula (I):
R₁—CO—O—R₂ (I)
wherein R₁ and R₂ are each independently chosen from a C₁-C₃₀, hydrocarbon chain optionally interrupted by at least one oxygen atom and/or by at least one carbonyl group, and optionally substituted by at least one hydroxyl group, and wherein R₁ is branched.

19. A multicompartment device comprising at least two compositions packaged separately, the mixing of which results in a composition comprising:
at least one peroxygenated salt; and
at least one non-volatile liquid branched ester of a carboxylic acid, the solidification point of which is below 4° C., and which is chosen from compounds of formula (I):
R₁—CO—O—R₂ (I)
wherein R₁ and R₂ are each independently chosen from a C₁-C₃₀, hydrocarbon chain optionally interrupted by at least one oxygen atom and/or by at least one carbonyl group, and optionally substituted by at least one hydroxyl group, and wherein R₁ is branched; and
hydrogen peroxide.

20. A device according to claim 19, wherein said device comprises:
a first compartment comprising a composition in the form of an anhydrous paste, said composition comprising:
at least one peroxygenated salt; and
at least one non-volatile liquid branched ester of a carboxylic acid, the solidification point of which is below 4° C., and which is chosen from compounds of formula (I):
R₁—CO—O—R₂ (I)
wherein R₁ and R₂ are each independently chosen from a C₁-C₃₀, hydrocarbon chain optionally interrupted by at least one oxygen atom and/or by at least one carbonyl group, and optionally substituted by at least one hydroxyl group, and wherein R₁ is branched; and
a second compartment comprising an aqueous composition comprising hydrogen peroxide.
